SEQUENCE LISTING (10 sequences)

SEQ ID NO: 1 - protein, 11 aa, Artificial
  Truncated version of a peptide found in colostrinin
QPLLQVMMEPQ

SEQ ID NO: 2 - protein, 6 aa, Artificial
  Truncated version of a peptide found in colostrinin
FLPVVN

SEQ ID NO: 3 - protein, 10 aa, Artificial
  Truncated version of a peptide found in colostrinin
PVLPVEPFPF

SEQ ID NO: 4 - protein, 10 aa, Artificial
  Truncated version of a peptide found in colostrinin
PKLKVEVPEP

SEQ ID NO: 5 - protein, 8 aa, Artificial
  Truncated version of a peptide found in colostrinin
ESYVPLFP

SEQ ID NO: 6 - protein, 7 aa, Artificial
  Truncated version of a peptide found in colostrinin
LLYQEPV

SEQ ID NO: 7 - protein, 9 aa, Artificial
  Truncated version of a peptide found in colostrinin
QPKVLPVPQ

SEQ ID NO: 8 - protein, 11 aa, Artificial
  Truncated version of a peptide found in colostrinin
PLPPTVMFPPQ

SEQ ID NO: 9 - protein, 9 aa, Artificial
  Truncated version of a peptide found in colostrinin
PVVVPPFLQ

SEQ ID NO: 10 - protein, 8 aa, Artificial
  Truncated version of a peptide found in colostrinin
LPLPLVRS

(19) United States
(12) Patent Application Publication
(10) Pub. No.: US 2004/0171553 A1
(43) Pub. Date: Sep. 2, 2004

(54) Peptides fragments of colostrinin and their use

(76) Inventor: Jerzy A. Georgiades, Houston, TX (US)

Correspondence Address: RANKIN, HILL, PORTER & CLARK, LLP, 925 EUCLID AVENUE, SUITE 700, CLEVELAND, OH, 44115-1405, US

(21) Appl. No.: 10/182,110

(22) PCT Filed: Jan. 26, 2001
(86) PCT No.: PCT/GB01/00329

(30) Foreign Application Priority Data
Jan. 26, 2000 (GB) 0001825.9

(51) Int. Cl. A61K 38/10; C07K 7/08

(52) U.S. Cl. 514/14; 530/327

(57) ABSTRACT

Fragments of colostrinin. These peptides are useful, inter alia, in the treatment of disorders of the immune system and the central nervous system, and are also useful as food additives.

The present invention relates to peptides. The invention also relates to therapeutic uses of the peptides and to antibodies derived therefrom.

In our International patent application WO00/75173 we have described a number of peptides present in colostrinin. We have now found a number of useful peptides which are derived from the peptides present in colostrinin.

According to one aspect of the present invention there is provided a peptide of having one of the following amino acid sequences 1-10:

| | | |
|---|---|---|
| 1. | QPLLQVMMEPQ | |
| 2. | FLPVVN | |
| 3. | PVLPVEPFPF | |
| 4. | PKLKVEVPEP | |
| 5. | ESYVPLFP | |
| 6. | LLYQEPV | (Position 204-210) |
| 7. | QPKVLPVPQ | (Position 182-200) |
| 8. | PLPPTVMFPPQ | (Position 165-175) |
| 9. | PVVVPPFLQ | (Position 97-105) |
| 10. | LPLPLVRS | (Position 150-157) |

These peptides may be provided in substantially isolated form. Furthermore, a composition may be provided which contains two or more of the above peptides, in combination.

The peptides 1 and 4 do not have any presently known precursor protein. The peptides 2, 3 and 5 are believed to have a β-casein homologue precursor. The peptides 6 to 10 generally correspond to a part of the peptide sequence for β-casein (the position on the β-casein amino acid chain being stated in brackets after each peptide).

In respect of the peptides 1 to 10, the invention further includes any peptide which includes an amino-terminal amino acid sequence corresponding to the specified sequence. Thus, with reference to peptide 1, for example, the invention encompasses any peptide having the N-terminal amino acid sequence QPLLQVMMEPQ; the same applies to peptides 2 to 10. For the avoidance of doubt, it is stated that the amino-terminal end is on the left hand side of the sequence, in accordance with the usual convention. It will be appreciated that any of the specified amino acid sequences may be provided with an inert amino acid sequence on the amino-terminal and/or the carboxy-terminal end thereof. The invention further includes physiologically acceptable active derivatives and analogs of the peptides. The polypeptides 1-10 can be in their free acid form or they can be amidated at the C-terminal carboxylate group. The present invention also includes analogs of the polypeptides 1-10, which includes polypeptides having structural similarity with peptides 1-10.

The peptides can be obtained by a number of techniques. In one embodiment, they are prepared by a conventional technique for peptide synthesis, such as by solid-phase or liquid-phase peptide synthesis. Alternatively, the gene sequence encoding the peptides can be constructed by known techniques such as expression vectors or plasmids and transfected into suitable microorganisms that will express the DNA sequences, whereby the peptides can be later extracted from the medium in which the microorganisms are grown. Thus, the invention also embraces a DNA sequence encoding the peptides described above, and a recombinant vector prepared by inserting said DNA in a vector.

The peptides, either alone or in combination with one another, have a number of therapeutic uses.

In one advantageous embodiment, one or more of peptides 1 to 10 may be used in the treatment of disorders of the central nervous system, particularly chronic disorders of the central nervous system. The disorders of the central nervous system that may be treated include neurological disorders and mental disorders. Examples of neurological disorders that may, with advantage, be treated include dementia, and also disorders that cause dementia, such as neurodegenerative disorders. Neurodegenerative disorders include, for example, senile dementia and motor neurone disease; Parkinson's disease is an example of a motor neurone disease that can be treated. Alzheimer's disease is an example of a neurodegenerative disease that can be treated. Examples of mental disorders that can be treated by one or more of the peptides include psychosis and neurosis. For example, the peptides may be used to treat emotional disturbances, especially the emotional disturbances of psychiatric patients in a state of depression. The peptides may also be used as an auxiliary withdrawal treatment for drug addicts, after a period of detoxification, and in persons dependent on stimulants.

In another advantageous embodiment of the invention, one or more of peptides 1 to 10 may be used in the treatment of disorders of the immune system, particularly chronic disorders of the immune system that may occur spontaneously in people of advanced age. The peptides can also be used in the treatment of diseases requiring immuno-modulation. The peptides are useful in the treatment of a variety of diseases with an immunological and infectious basis. For example, they can be used to treat chronic diseases with a bacterial and viral aetiology, and to treat acquired immunological deficiencies that have developed, for example, after chemotherapy or radiotherapy of neoplasms. The peptides may be used for treating chronic bacterial and viral infections requiring non-specific immunostimulation and immunocorrection.

A chronic disorder is a disorder that has persisted, or is expected to persist, for a long time, i.e., at least 3 months and usually at least 6 months.

One or more of the peptides may be used for improving the development of the immune system of a new born child. It is a further feature of the invention to use the peptides to correct immunological deficiencies in a child. These uses of the peptides may be particularly applicable to babies or children who have been deprived of colostrum. This may occur, for example, in babies and children who were not breast fed from birth.

The peptides, either alone or in combination with one another, also have diagnostic and research applications. For example, the synthetic peptides, as well as the corresponding antibodies described below, may be used to recognise pathological processes occurring in a host. These processes may be induced by excessive production or inhibition of the peptides or the antibodies. Once the pathological process associated with a particular level of the peptides or the antibodies is known, measuring the production of the peptides and the antibodies in body fluids may be used to determine pathological processes taking place in the host.

According to another aspect of the invention, we provide the use of one or more of peptides 1 to 10 as a dietary supplement. This dietary supplement is particularly useful for babies, especially premature babies and babies at term, and for young children to correct deficiencies in the development of their immune system. The dietary supplement may also be used as a dietary supplement for adults, including senile persons, who have been subjected to chemotherapy, or have suffered from cahexia, or weight loss due to chronic disease.

In an aspect of the invention, we provide a dietary supplement comprising an orally ingestible combination of one or more of peptides 1 to 10 in combination with a physiologically acceptable carrier. The dietary supplement may be provided in liquid or solid form; the dietary supplement may suitably be provided in the form of a tablet. The dietary supplement may be provided in the form of a baby food formula. The dietary supplement may include, as an additive, lactoferrin and/or selenium and/or a group of cytokines containing members of the interferon family.

In accordance with the invention, one or more of peptides 1 to 10 may be administered prophylactically in order to help to prevent the development of disorders of the central nervous system and the immune system.

The peptides 1 to 10 promote the dissolution of certain parts of the beta-amyloid precursor protein, which is now believed to be present in the brain in the form of plaque depositions in patients with Alzheimer's disease. Thus the invention also embraces the use of the peptides 1 to 10 to dissolve the beta-amyloid precursor protein and to dissolve the beta-amyloid peptide itself. In principle, it is believed that the peptides 1 to 10 may be used in the treatment of any disease which is characterised by the development of β-amyloid plaques.

The peptides according to the invention may be administered in a dosage in the range 1 ng to 10 mg. A dosage unit of about 3 μg is typical. However, the optimum dosage will, of course, depend upon the condition being treated.

The peptides according to the invention may be formulated for administration in any suitable form, although they are preferably formulated as an oral dosage form. Thus, the invention further provides a composition, especially a pharmaceutical composition, which includes one or more of the peptides in combination with a physiologically acceptable carrier. The peptides may, for example, be formulated for oral, topical, rectal or parenteral administration. More specifically, the peptides may be formulated for administration by injection, or, preferably, in a form suitable for absorption through the mucosa of the oral/nasopharyngeal cavity, the alimentary canal or any other mucosal surface. The peptides may be formulated for administration intravenously, subcutaneously, or intramuscularly. The oral formulations may be provided in a form for swallowing or, preferably, in a form for dissolving in the saliva, whereby the formulation can be absorbed in the mucous membranes of the oral/nasopharyngeal cavity. The oral formulations may be in the form of a tablet for oral administration, lozenges (i.e. a sweet-like tablet in a form suitable to be retained in the mouth and sucked), or adhesive gels for rubbing into the gum. The peptides may be formulated as an adhesive plaster or patch, which may be applied to the gums. The peptides may also be formulated for application to mucous-membranes of the genito-urinary organs. The topical formulations may be provided in the form of, for example, a cream or a gel.

One or more of the peptides may be incorporated into products like milk or cheese spread.

In another aspect, the invention provides an antibody for each of the peptides 1 to 10, and provides compositions containing said antibodies. In particular the invention provides the antibodies in substantially isolated form. The antibodies can be produced by injecting a suitable mammalian subject, such as a rabbit, with the corresponding peptide (with a suitable adjuvant), then recovering the antibodies from the subject after allowing time for them to be produced. This technique is described in Example 2 below, and can be used to form antibodies to the peptides 1 to 10. It is possible to test that the correct antibody has been produced by ELISA (enzyme-linked immunosorbent assay) using the synthetic peptides as antigens. The antibodies have potential uses in therapy, as a diagnostic tool and as a research tool.

The invention also encompasses the selective administration of one or more of peptides 1 to 10, at selected times to a patient, and the selective administration of one or more of the antibodies for the peptides in order to switch on or off the activity of the peptides at a selected time.

A selection of selected ones of the peptides and/or antibodies may be provided in a single composition which is specially tailored to produce a particular effect. For example, for a person with an immunological disorder, the composition can be specially tailored for that disorder. The composition may be specially selected for more than one disorder. The composition may be specially selected to restore or produce a particular balance in a subject.

In some applications it may be desirable to provide a pharmaceutical composition which contains one or more of the peptides and one or more of the antibodies in combination with a physiologically acceptable carrier.

The invention further embraces the use of one or more of the peptides and/or antibodies in the manufacture of a medicament for use in any of the therapeutic applications described above.

Reference is now made to the accompanying drawings, in which:

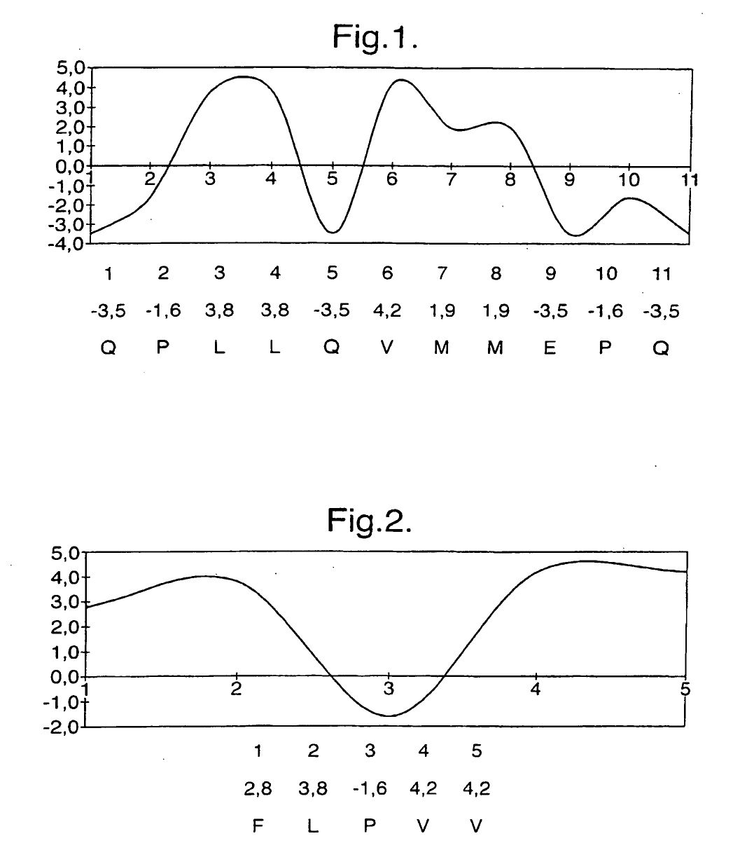

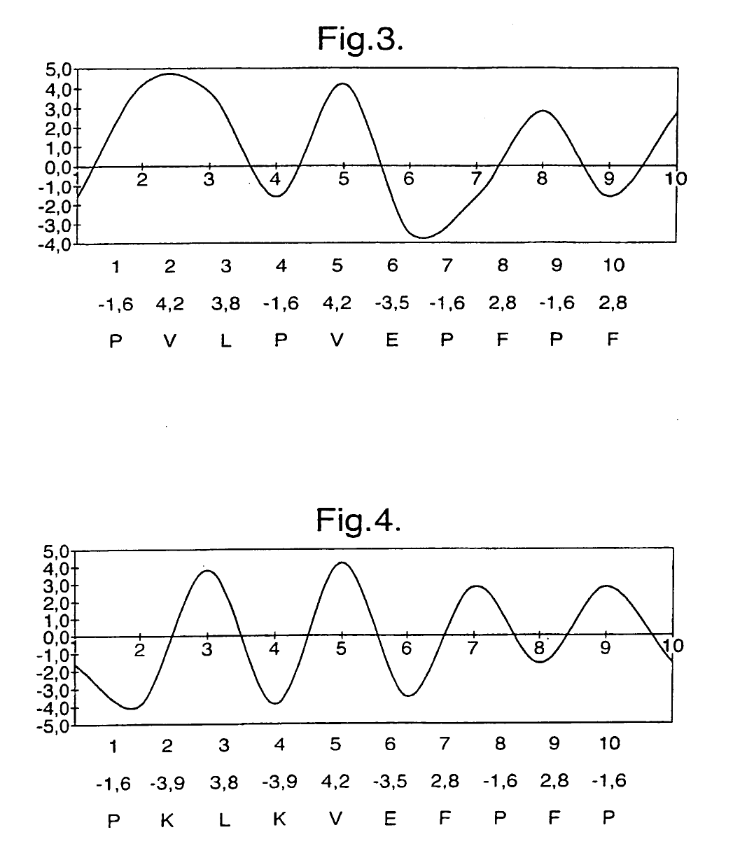

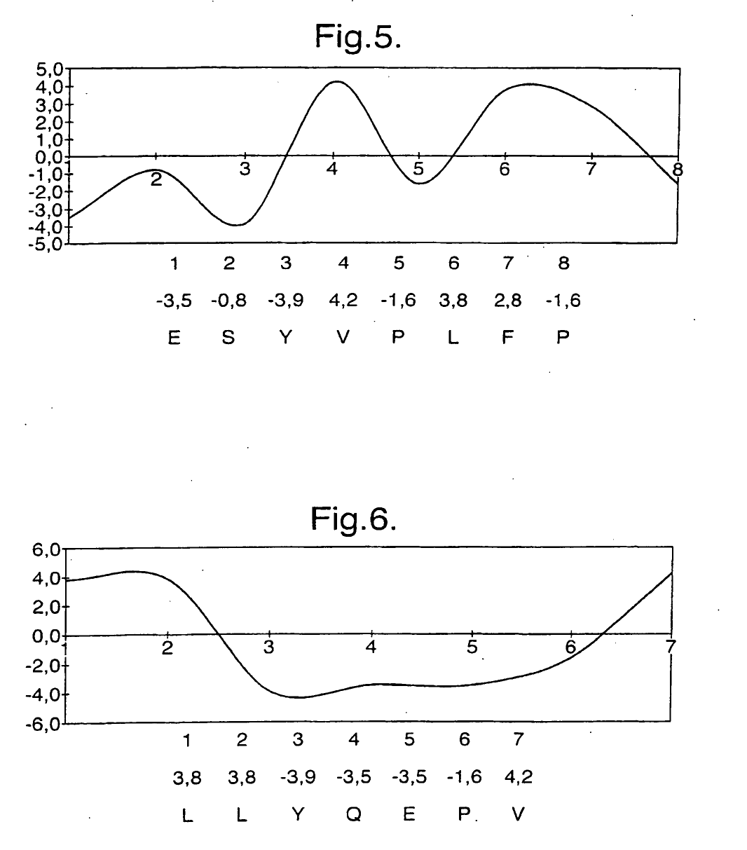

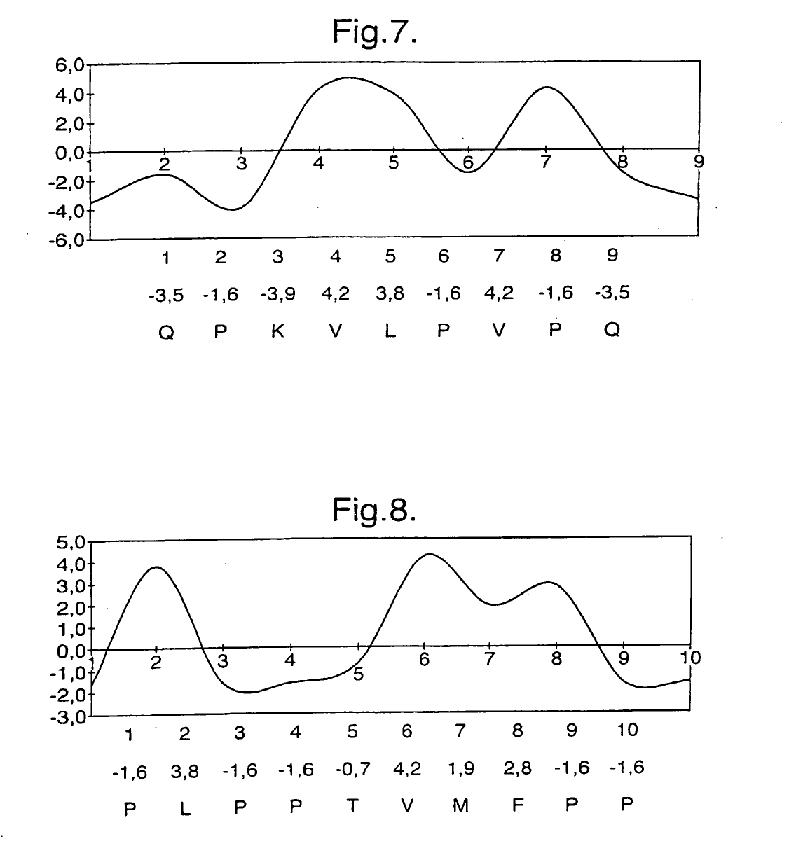

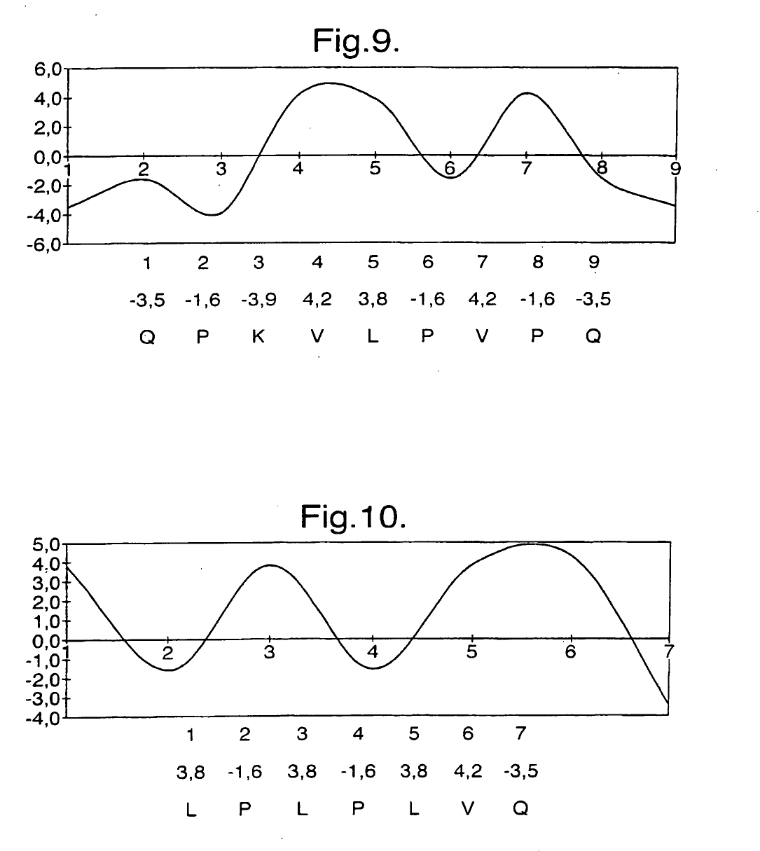

FIGS. 1 to 10 are graphs of hydrophobicity against amino acid for each of the peptides 1 to 10 respectively; and

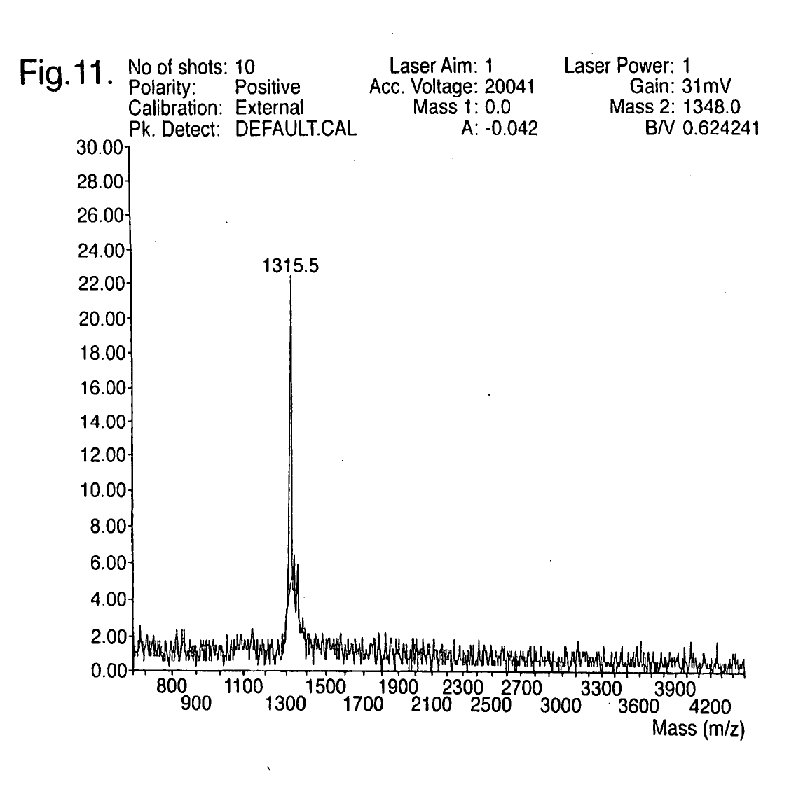

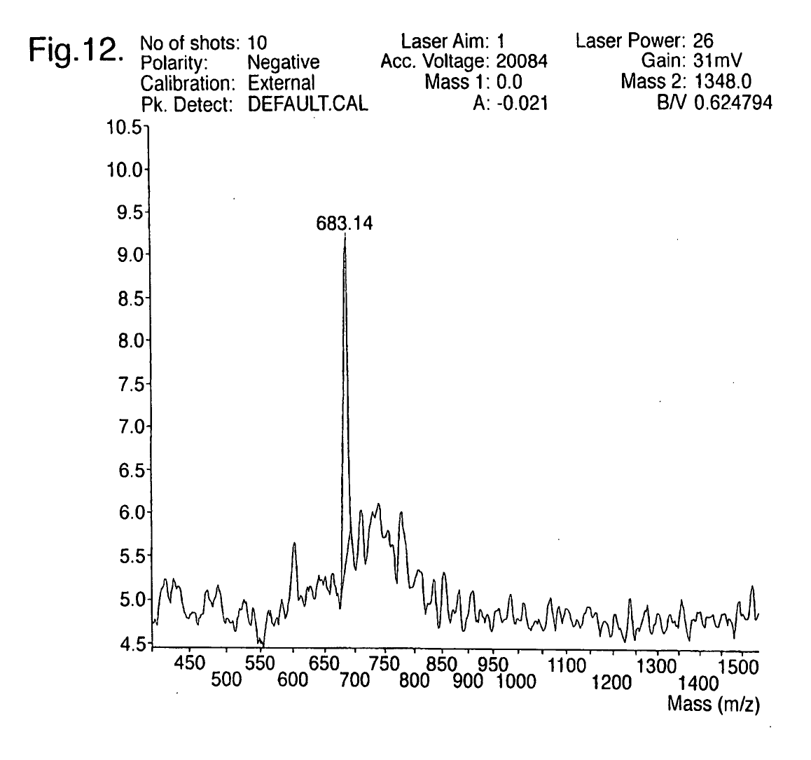

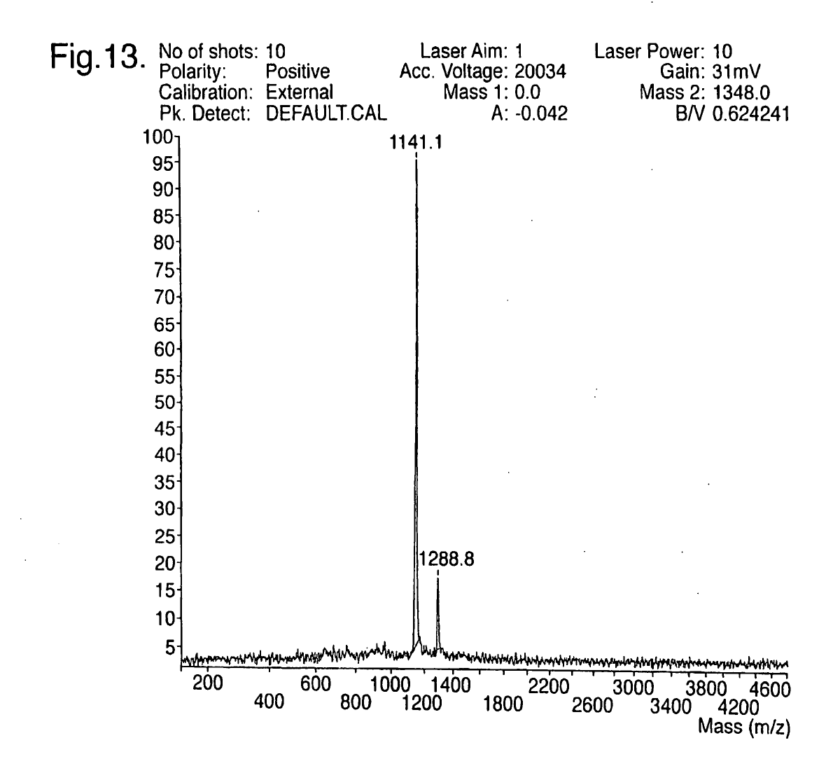

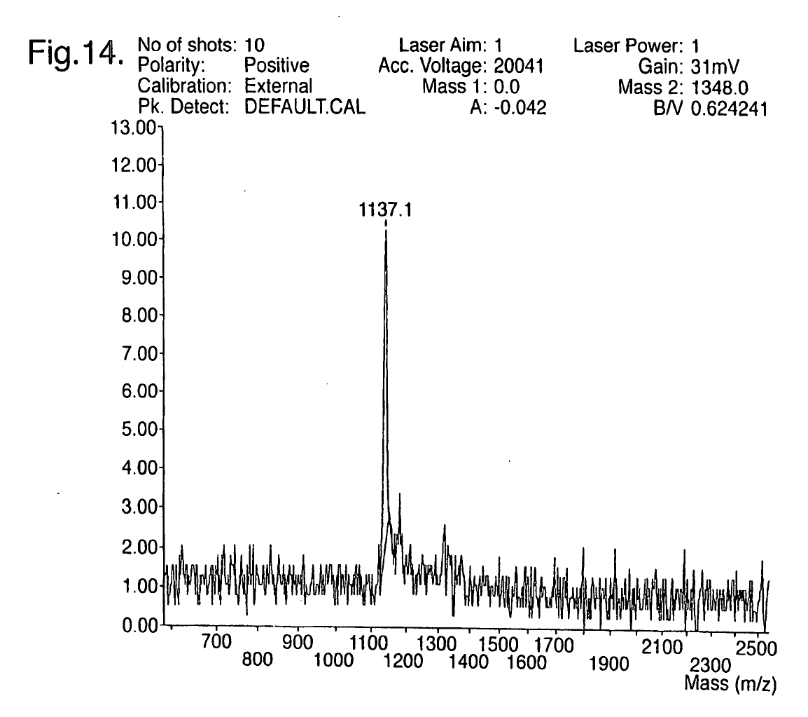

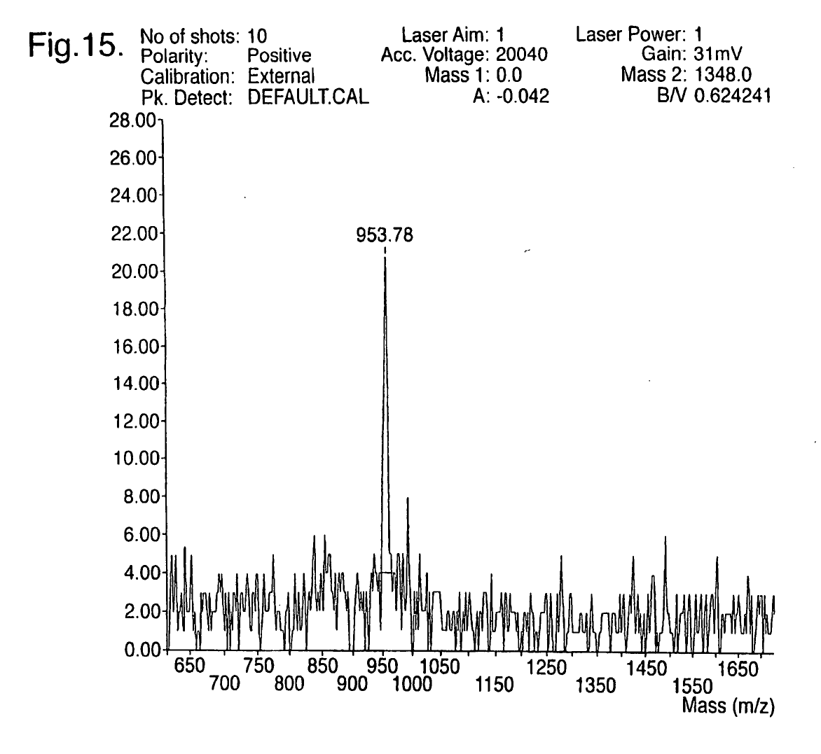

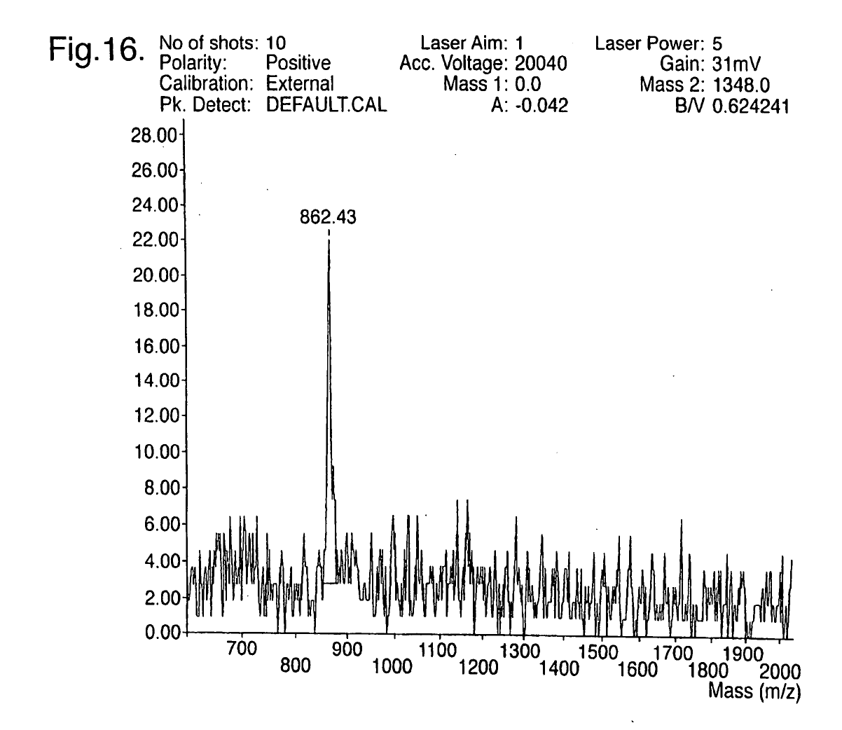

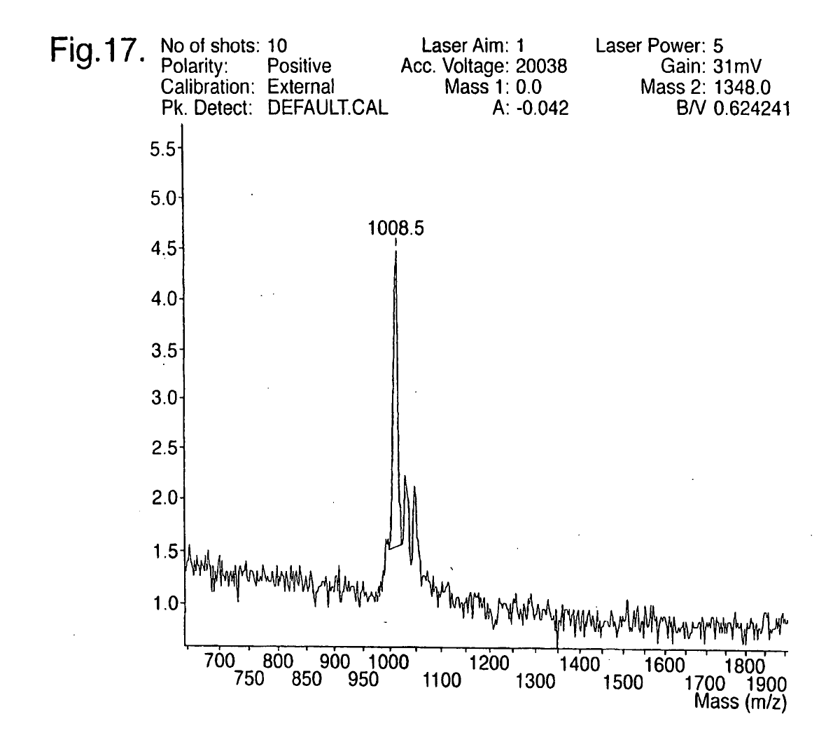

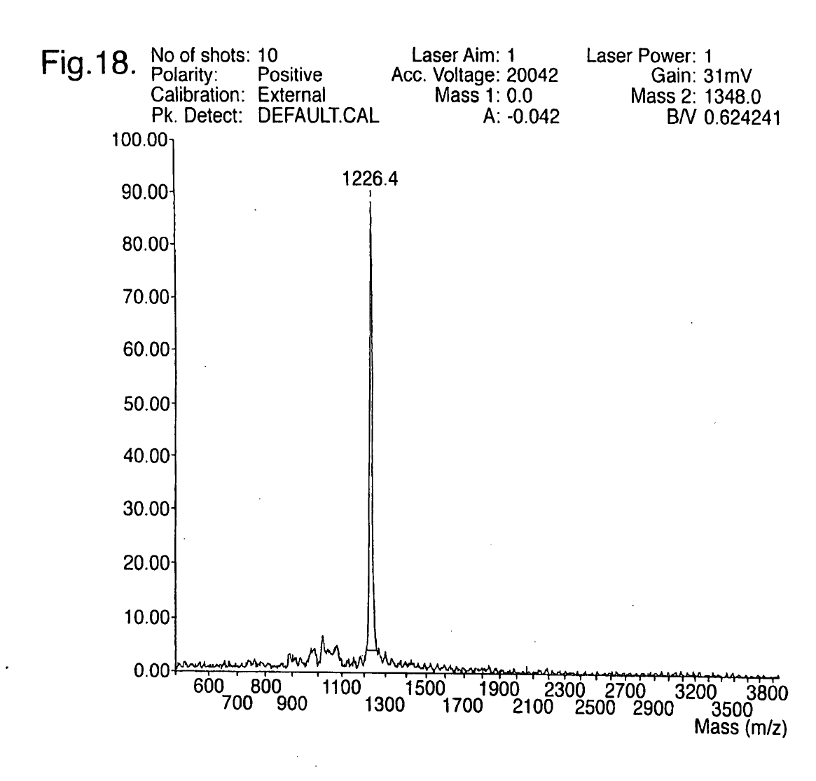

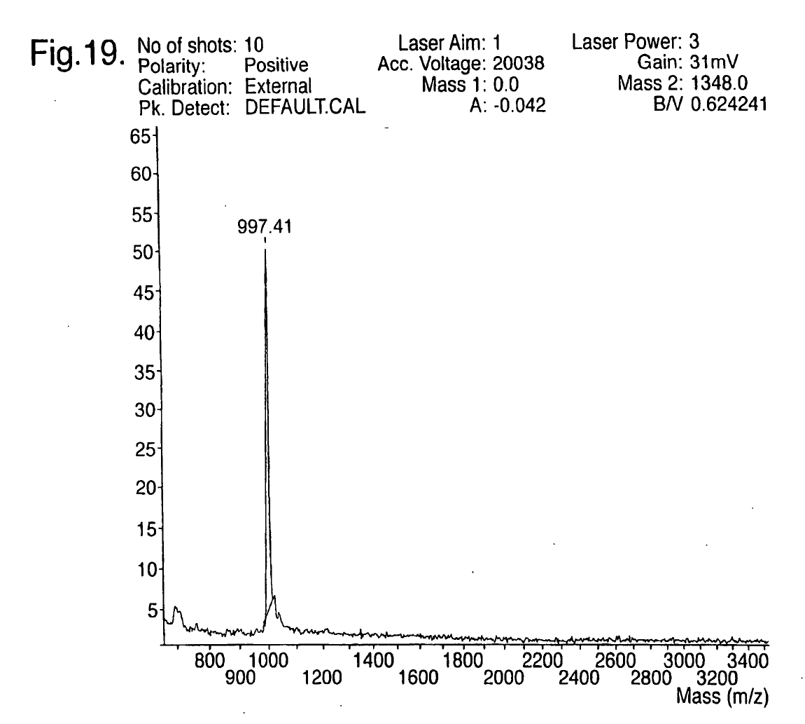

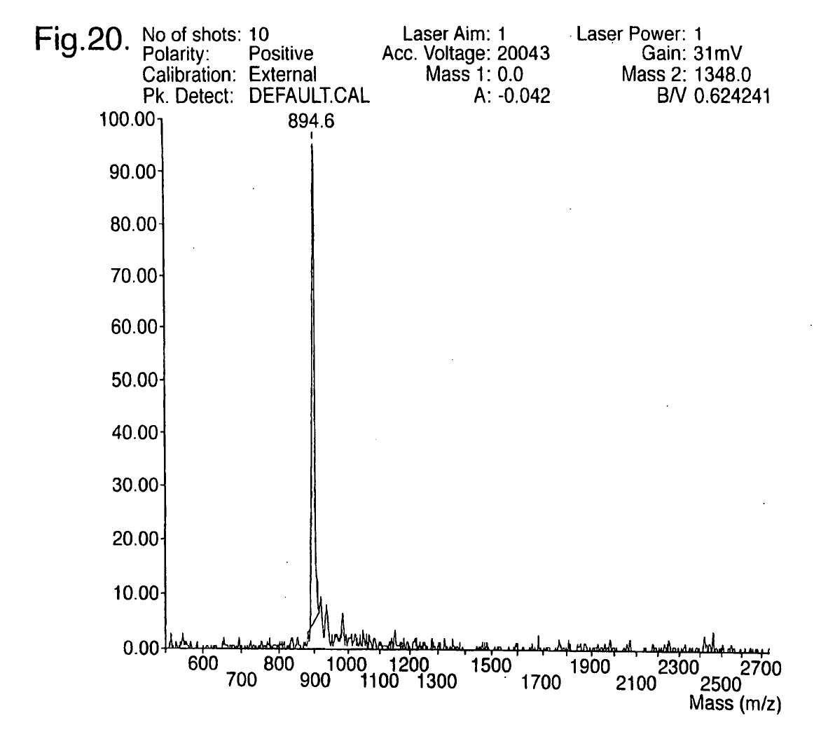

FIGS. 11 to 20 are Matrix-Assisted Laser Desorption Time-of-Flight Mass Spectroscopy (LDMS) spectra of certain peptides according to the invention.

FIGS. 1 to 10 show the way the hydrophobicity of the peptides 1 to 10 varies along each peptide. The invention encompasses peptides having substantially the hydrophobicity profiles shown in the drawings.

The invention will now be further described with reference to the following examples.

### EXAMPLE 1

### Production of the Peptides

The peptides identified in example 1-10 were produced by the synthetic technique known as the solid phase method. This method involved the following steps:

1. Wash pre-loaded resin with DMF (dimethylformamide), then drain completely.

2. Add 10 ml of 20% piperidine/DMF to resin. Shake for 5 mins, then drain.

3. Add another 10 ml of 20% piperidine/DMF. Shake for 30 mins.

4. Drain reaction vessel and wash resin with DMF four times. Then wash once with DCM (dichloromethanol). Check beads using the ninhydrin test

the beads should be blue.

5. The coupling step was carried out as follows:

Prepare the following solution:

1 mmole Fmoc (i.e. fluorenylmethyloxycarbonyl) amino acid 2.1 ml of 0.45 M HBTU/HOBT (1 mmol) (2-(1H-benzotriazol-1-yl)-1,1,3,3-tetramethyluronium hexafluorophosphate/N-hydroxybenzotriazole-H₂O) 348 μl of DIEA (2 mmol) (diisopropylethylamine)

Add the solution to the resin and shake for a minimum of 30 minutes.

6. Drain reaction vessel and wash the resin again with DMF four times and with DCM once.

7. Perform the ninhydrin test:

If positive (no colour)—proceed to step 2 and continue synthesis.

If negative (blue colour)—return to step 5 and recouple the same Fmoc amino acid.

8. After the synthesis was complete, the peptide was cleaved from the resin with 5% H₂O, 5% phenol, 3% Thionisole, 3% EDT (ethanedithiol), 3% triisopropylsilane and 81% TFA for 2 hours.

9. After 2 hours, filter into cold MTBE (methyl t-butyl ether). The precipitated peptide was then washed twice with cold MTBE and dried under nitrogen gas.

10. The molecular weight of the synthesised peptides was checked by Matrix-Assisted Laser Desorption Time-of-Flight Mass Spectroscopy (LDMS). and the purity was checked by hplc using a C-18, 300 Angstrom, 5 μm column. The resulting spectra of some peptides are shown in FIGS. 11 to 20.

Certain structural data about the peptides is shown in Table 1.

TABLE 1

| | Hydrophobicity | | Mass Sp. | |
|---|---|---|---|---|
| Peptide | Index | M.W. | M.W | HPLC el. time |
| 1 | −2.1 | 1314.6 | 1315.5 | 9.65 |
| 2 | +9.9 | 686.8 | 683.14 | 8.83 |
| 3 | +8.7 | 1142.39 | 1141.1 | 10.8 |
| 4 | −7.8 | 1136.4 | 1137.1 | 8.22 |
| 5 | +3.1 | 952.1 | 953.78 | 10.12 |
| 6 | +1.9 | 862.0 | 862.43 | 8.67 |
| 7 | −3.5 | 1006.2 | 1008.5 | 8.04 |
| 8 | +0.5 | 1224.5 | 1226.4 | 9.42 |
| 9 | +10.9 | 996.2 | 992.41 | 10.10 |
| 10 | +7.1 | 895.1 | 894.6 | 9.02 |

The mass spectrometry results show that the measured molecular weight is to the actual molecular weight, i.e., that the correct peptides have been formed.

### EXAMPLE 2

### Preparation of the Antibodies

To each N-terminal end of the synthetic peptides, L-cysteine was attached, and the peptide was formed into a ring so that the cysteine group lay between the N-terminal and the C-terminal ends of the synthetic peptide. This facilitated peptide conjugation with Keyhole Hemolymph (KHL).

TABLE 2

| | ORIGINAL | |
|---|---|---|
| PEPTIDE SYNTHESISED | PEPTIDE | FIGURE NO. |
| NH₂-(Ac)CQPLLQVMMEPQ-OH | 1 | 1 |
| NH₂-(Ac)CFLPVVN-OH | 2 | 2 |
| NH₂-(Ac)CPVLPVEPFP-OH | 3 | 3 |
| NH₂-(Ac)CPKLKVEVPEP-OH | 4 | 4 |
| NH₂-CESYVPLFP-OH | 5 | 5 |
| NH₂-(Ac)CLLYQEPV-OH | 6 | 6 |
| NH₂-(Ac)CQPKVLPVPQ-OH | 7 | 7 |
| NH₂-(Ac)CPLPPTVMFPPQ-OH | 8 | 8 |
| NH₂-(Ac)CPVVVPPFLQ-OH | 9 | 9 |
| NH₂-CLPLPLVRS-OH | 10 | 10 |

The invention further provides each of the peptides specified in Table 2, and the cyclisised version of each of these peptides, especially in isolated form and produced by a synthetic process. The term “Ac” represents an acyl group.

For immunisation, two young adult rabbits (5-6 months old, weighing 5-6 lbs [2.3-2.7 kg]) were used. Each antigen (i.e., each synthetic peptide) was given subcutaneously and intramuscularly in 0.1 ml injections at ten different sites. The protocol used followed the following sequence:

| Day | Procedure |
|---|---|
| 0 | Prebleed & initial inoculation of rabbit with 200 μg of the peptide |
| | at 0.5 ml of conjugate solution mixed with an equal volume of |
| | complete Freund's adjuvant (mineral oil/emulsifier/killed |
| | mycobacteria). |
| 14 | Boost inoculation with 200 μg of the peptide at 0.5 ml of conjugate |
| | solution mixed with an equal volume of incomplete Freund's |
| | adjuvant (mineral oil/emulsifier). |
| 28 | Boost (as on day 14) |
| | Production Bleed (approx. 20 ml sera) |
| 42 | Boost (as on day 14) |
| | Production Bleed (approx. 20 ml sera) |
| 56 | Boost (as on day 14) |
| | Production Bleed (approx. 20 ml sera) |
| 70 | Boost (as on day 14) |

### Production Bleed (Approx. 20 ml Sera)

This protocol may be varied. For example, the frequency of the production bleed depends upon, inter alia, the size and health of the host species.

The sera produced by this protocol were used for IgG purification on a Protein A matrix (from Sigma, based in St. Louis, Mo., USA). The protocol was as follows:

1. Wash columns with 10 ml 1×PBS (phosphate buffered saline). There were two 1 m column arranged in tandem each containing the Protein A matrix.

2. Add 3 ml of the serum to 3 ml of PBS and divide this mixture between the two columns.

3. Collect the serum into a test tube as it drains through the column.

4. When the serum finishes draining, pour the washed serum back into the column and begin collecting flow through again. Repeat this step 5 to 6 times.

5. Wash the columns with 10 ml of 1×PBS.

6. Prepare several 1 ml tubes with 50 μl of 1 M TRIS (2-amino-2-hydroxymethyl-1,3-propanediol) (pH=9,5).

7. Add 1 ml of elution buffer (100 mM glycine, pH=2.8) to each tube and collect 1 ml of flow therethrough.

8. Move to the next prepare tube and repeat step 7.

9. Test each 1 ml sample by preparing ELISA plate with 10 μl of Bradford Assay and add 50 μl of each 1 ml flow through. Keep the samples that change the Bradford Assay from red to blue.

10. Dialyse the positive 1 ml samples together in 4 litres of 1×PBS at pH=7.2 for at least 24 hours.

11. Use spectrometer at 280 nm to find concentration of IgG in solution (extinction coefficient=1.4).

12. To store IgG solution, keep frozen at −4° C. to −20° C.

The level of antibodies in the serum was established by ELISA (enzyme-linked immunosorbent assay) with the corresponding synthetic peptide antigen. This technique involved the following steps:

1. The antigen was diluted with a 0.1 M bicarbonate buffer (pH 9.0) to yield a 10 μg of antigen/ml solution. A volume of 50 μl of this solution was placed into each microwell of a 96 well plate.

2. The plates were covered and incubated at 37° C. for 3 hours.

3. The wells were washed with a coupling buffer and blocked using 200 μl of Pierce standard solution of BSA (bovine serum albumin).

4. 50 μl of dilutent BSA (0.75% soln.) was pipetted into each well. 50 μl of antibody serum sample diluted 1:100 in dilutent BSA were placed in lane A of each row.

5. 1:2 serial dilutions were performed moving down the plate.

6. The plates were covered and incubated at room temperature for 60 minutes.

7. The plates were washed four times with PBS wash solution.

8. A volume of 50 μl of goat anti-rabbit IgG (H&L) HRP conjugate at 1:1000 dilution in BSA was pipetted into each well and incubated at room temperature for 60 minutes (H&L=heavy and light chain; HRP=horseradish peroxidase).

9. The plates were washed four times with PBS wash solution.

10. A volume of 50 μl of substrate solution 2,2′-azino-bis(3-ethylbenzothiazoline-6-sulfonic acid)-diammonium salt (ABTS available from Pierce, which is used to help visualise the extent of the antibody/antigen reaction) was pipetted into each well and incubated at room temperature for about 2 minutes.

11. The reaction was stopped by adding 50 μl of 1% SDS (sodium dodecyl sulfate) into each well.

12. The wells were then read on a dynoplate reader at 405.

The data presented in Table 3 show the serum antibody titers against specific antibodies after the 10 week immunisation protocol.

TABLE 3

Titre: (Serum Dilution)

| | |
|---|---|
| Pre | Post |
| Immunization | Immunization |

| | | | | | |
|---|---|---|---|---|---|
| No | Sequence | R1 | R2 | R1 | R2 |

| | | | | | |
|---|---|---|---|---|---|
| 1 | QPLLQVMMEPQ | 0 | 0 | 12800 | 25600 |
| 2 | FLPWN | 0 | 0 | 12800 | 12800 |
| 3 | PVLPVEPFPF | 0 | 0 | 12800 | 12800 |
| 4 | PKLKVEVPEP | 0 | 0 | 25600 | 25600 |
| 5 | ESYVPLFP | 0 | 0 | 25600 | 25600 |
| 6 | LLYQEPV | 0 | 0 | 25600 | 25600 |
| 7 | QPKVLPVPQ | 0 | 0 | 25600 | 25600 |
| 8 | PLPPTVMFPPQ | 0 | 0 | 25600 | 25600 |
| 9 | PVVVPPFLQ | 0 | 0 | 25600 | 25600 |
| 10 | LPLPLVRS | 0 | 0 | 25600 | 25600 |

### EXAMPLE 3

Ischemia was induced in rats in order to encourage the development of the beta-amyloid precursor proteins. The rats were then subjected to a variety of treatment regimes, and the degree of the beta-amyloid precursor protein presence was investigated. The general technique is described in “Complete cerebral ischemia with short-term survival in rats induced by cardiac arrest. I. Extracellular accumulation of Alzheimer's β-amyloid protein precursor in the brain.”, Pluta, R. et al, Brain Research 649 (1994) 323-328.

The treatment regime varied in terms of the length of time ischemia was induced, and the length of time treatment was applied.

The Colostrinin was obtained by conventional techniques, as described, for example, in WO98/14473. The peptides were produced using the method described in Example 1. The control was not subjected to any treatment.

The results are shown in the Table 4 below. These results show the β-amyloid plaque level corresponding to the N-terminal end of the precursor protein, the C-terminal end of the precursor protein, and the 511-608 position of the precursor protein. Each column represents the results if the immunostaining with specific monoclonal antibodies directed against fragments of amyloid or its N and C precursor protein. It will be seen that Colostrinin provided generally good results and peptides 1, 3 and 5 provided particularly good results.

TABLE 4

| | | 511-608 | Jonas | |
|---|---|---|---|---|
| | Beringer- | APP | C-terminal | |
| Peptide | terminal APP | ALZ 90 | APP | Total |
| Colostrinin control | 0.3 | 0 | 0.3 | 0.6* |
| Colostrinin control | 1.7 | 0.3 | 1 | 3* |
| Colostrinin control | 1.7 | 1.6 | 0.3 | 3.6* |
| Colostrinin control | 0.6 | 1.6 | 1 | 3.2* |
| Colostrinin 2 | 2 | 4 | 2 | 8* |
| Colostrinin 3 | 3 | 2 | 3 | 8* |
| Colostrinin 4 | 2 | 2 | 3 | 7* |
| Placebo Control | 3.5 | 3.8 | 2 | 9.3** |
| Peptide 1 | 0 | 0 | 1 | 1 |
| Peptide 1 | 0 | 0 | 2 | 2 |
| Peptide 1 | 1 | 2 | 2 | 5 |
| Peptide 3 | 0 | 3 | 1 | 4 |
| Peptide 3 | 2 | 3 | 2 | 7 |
| Peptide 3 | 0 | 0 | 2 | 2 |
| Peptide 5 | 2 | 5 | 3 | 9 |
| Peptide 5 | 2 | 0 | 2 | 4 |
| Peptide 5 | 4 | 3 | 1 | 8 |
| Peptide 7 | 4 | 4 | 3 | 11 |
| Peptide 7 | 3 | 4 | 3 | 10 |
| Peptide 7 | 1 | 5 | 3 | 9 |
| Peptide 9 | 2 | 3 | 3 | 8 |
| Peptide 9 | 1 | 2 | 2 | 5 |
| Peptide 9 | 3 | 1 | 2 | 6 |

*Different protocol of administration
**Control animals without any treatment other than induction of ischemia

It will be appreciated that the invention described above may be modified.

1. A peptide which substantially includes the amino-terminal amino acid sequence: QPLLQVMMEPQ (SEQ ID 1); FLPWN (SEQ ID 2); PVLPVEPFPF (SEQ ID 3); PKLKVEVPEP (SEQ ID 4); ESYVPLFP (SEQ ID 5); LLYQEPV (SEQ ID 6); QPKVLPVPQ (SEQ ID 7); PLPPTVMFPPQ (SEQ ID 8); PVVVPPFLQ (SEQ ID 9); LPLPLVRS (SEQ ID 10).

2. A peptide which substantially entirely consists of the amino acid sequence: QPLLQVMMEPQ (SEQ ID 1); FLPVWN (SEQ ID 2); PVLPVEPFPF (SEQ ID 3); PKLKVEVPEP (SEQ ID 4); ESYVPLFP (SEQ ID 5); LLYQEPV (SEQ ID 6); QPKVLPVPQ (SEQ ID 7); PLPPTVMFPPQ (SEQ ID 8); PVVVPPFLQ (SEQ ID 9); LPLPLVRS (SEQ ID 10).

3. A peptide according to claim 1 or 2 in substantially isolated form.

4. A peptide according to claim 1, 2 or 3, when obtained by a synthetic process.

5. A peptide according to any preceding claim, for use as a medicament.

6. A peptide according to claim 5, for use in the treatment of chronic disorders of the central nervous system.

7. A peptide according to claim 5, for use in the treatment of neurological disorders and/or mental disorders.

8. A peptide according to claim 5, for use in the treatment of dementia and/or neurodegenerative diseases.

9. A peptide according to claim 5, for use in the treatment of Alzheimer's disease and/or motor neurone disease.

10. A peptide according to claim 5, for use in the treatment of psychosis and/or neurosis.

11. A peptide according to claim 5, for use in the treatment of chronic disorders of the immune system.

12. A peptide according to claim 5 for use in the stimulation and/or modulation of the immune system.

13. A peptide according to claim 5, for use in the treatment of diseases with a bacterial and viral aetiology, and/or for use in the treatment of acquired immunological deficiencies.

14. A peptide according to claim 5, for use in the treatment of chronic bacterial and/or viral infections.

15. A peptide according to claim 5, for use in the treatment of diseases characterised by the presence of β-amyloid plaque.

16. The use of a peptide according to any one of claims 1 to 4, in the manufacture of a medicament for the treatment of chronic disorders of the central nervous system.

17. The use of a peptide according to any one of claims 1 to 4 in the manufacture of a medicament for the treatment of chronic disorders of the immune system.

18. A method of treating disorders of the central nervous system and/or of the immune system, comprising administering a therapeutically effective amount of a peptide according to any one of claims 1 to 4 to a patient.

19. A pharmaceutical composition comprising a peptide according to any one of claims 1 to 4, in combination with a physiologically acceptable carrier.

20. A composition comprising two or more peptides according to any one of claims 1 to 4, in combination with a physiologically acceptable carrier.

21. A pharmaceutical composition according to claim 19 or 20, in a form suitable for injection.

22. A pharmaceutical composition according to claim 19 or 20, in a form suitable for absorption through the mucosa of the oral/nasopharyngeal cavity and/or in a form suitable for absorption in the alimentary canal.

23. A composition according to claim 19 or 20, in the form of a tablet, lozenge, gel, patch or plaster.

24. A composition according to claim 19 or 20, in a form suitable for topical application.

25. The use of a peptide according to any one of claims 1 to 4 as a dietary supplement.

26. The use of a peptide according to any one of claims 1 to 4 as a dietary supplement for babies, small children, adults who have been subjected to chemotherapy and/or adults who have suffered from cahexia, or weight loss due to chronic disease.

27. A dietary supplement comprising an orally ingestible combination of a peptide according to any one of claims 1 to 4 in combination with a physiologically acceptable carrier.

28. An antibody which binds to a peptide according to any one of claims 1 or 4.

29. An antibody obtainable by using a peptide according to any one of claims 1 to 4 as an antigen.

30. A method of dissolving β-amyloid plaques comprising contacting the plaque with a peptide according to any one of claims 1 to 4.
